(19) United States
(12) Patent Application Publication
(10) Pub. No.: US 2026/0220404 A1
(43) Pub. Date: Jul. 30, 2026

(54) IDENTIFICATION METHOD AND ANALYTICAL METHOD OF DETECTING AT LEAST ONE ANALYTE IN A SAMPLE OF A BODY FLUID

(71) Applicant: Roche Diabetes Care, Inc., Indianapolis, IN (US)

(72) Inventors: Bernhard Limburg, Soergenloch (DE); Max Berg, Mannheim (DE)

(21) Appl. No.: 19/467,356

(22) Filed: Feb. 2, 2026

Related U.S. Application Data
(63) Continuation of application No. PCT/EP2024/071664, filed on Jul. 31, 2024.

(30) Foreign Application Priority Data
Aug. 3, 2023 (EP) 23 189 500.4

(51) Int. Cl. G06K 7/10; G06T 7/00; G06T 7/90; H03M 13/15

(52) U.S. Cl. CPC G06K 7/10366; G06T 7/0012; G06T 7/90; H03M 13/155; G06T 2207/10024

(57) ABSTRACT

An identification method configured for an analytical method of detecting at least one analyte in a sample of a body fluid is disclosed. The identification method uses a mobile device, specifically a mobile communication device, that has a camera and an RFID reader. In the identification method, a user is prompted to identify by bringing at least one RFID identifier of the user into a communication range of the RFID reader. Data from the RFID identifier obtained by using the RFID reader is read out and validated. The validation includes checking if a valid item of identification information of the user was successfully transferred with the data from the RFID identifier to the RFID reader. Further disclosed is a computer program and a computer-readable storage medium for performing the disclosed methods.

### RELATED APPLICATIONS

This application is a continuation of International Patent Application No. PCT/EP2024/071664, filed Jul. 31, 2024, which claims priority to EP 23 189 500.4, filed Aug. 3, 2023, both of which are hereby incorporated herein by reference.

### BACKGROUND

This disclosure refers to a computer-implemented identification method of identifying a user for an analytical method and to a computer-implemented analytical method for detecting at least one analyte in a sample of a body fluid of a user. (“Body fluid” and “bodily fluid” are used interchangeably herein.) This disclosure further refers to a computer program and a computer-readable storage medium for performing the methods. The methods may specifically be used in medical diagnostics in order to quantitatively or qualitatively detect one or more properties of a body fluid, e.g., for or qualitatively detecting the presence of SARS-CoV-2 in the sample of the body fluid or, alternatively, for quantitatively detecting an analyte, such as blood glucose, in the sample of the body fluid. Other fields of application of this disclosure, however, are also feasible.

In the field of medical diagnostics, devices and methods are known which make use of test elements comprising one or more test chemicals, which, in presence of an analyte to be detected, are capable of performing one or more optical detection reactions. The optically detection reactions may be evaluated using, e.g., cameras of mobile devices, such as a camera of a smartphone. Some of these mobile device-based diagnostics solutions may provide personalized test certificates and/or personalized test evidence on an outcome of the performed test for the user of the mobile device. Thus, the user running the test must generally be uniquely identified. As an example, for mobile device-based diagnostics solutions, identification may be performed using a camera-based identification method comprising the presentation of ID cards to dedicated human invigilators.

In general, methods for user identification in the medical and/or diagnostic field are known. For example, U.S. Publication No. 2022/0223300 A1, U.S. Pat. No. 11,289,196 B1 and U.S. Pat. No. 11,367,530 B1 describe systems and methods for providing a universal platform for at-home health testing and diagnostics. In particular, a health testing and diagnostic platform is described to connect medical providers with patients and to generate a unique, private testing environment. In some embodiments, the testing environment may facilitate administration of a medical test to a patient with the guidance of a proctor. In some embodiments, the patient may be provided with step-by-step instructions for test administration by the proctor within a testing environment. The platform may display unique, dynamic testing interfaces to the patient and proctor to ensure proper testing protocols and accurate test result verification.

U.S. Publication No. 2018/0353073 A1 discloses systems, tools and methods for acquiring diagnostic information, including medical information, for a user, transmitting the information to a remote location, assessing the information, and transmitting resulting diagnosis and treatment information to the user and/or a third party for subsequent action.

U.S. Pat. No. 10,892,052 B2 describes aspects of remote presence interfaces (RPis) for use on portable electronic devices (PEDs) to interface with remote presence devices. An RPI may allow a user to interact with a telepresence device, view a live video feed, provide navigational instructions, and/or otherwise interact with the telepresence device. The RPI may allow a user to manually, semi-autonomously, or autonomously control the movement of the telepresence device. One or more panels associated with a video feed, patient data, calendars, date, time, telemetry data, PED data, telepresence device data, healthcare facility information, healthcare practitioner information, menu tabs, settings controls, and/or other features may be utilized via the RPI.

U.S. Pat. No. 11,373,756 B1 describes an at-home medical diagnostic test kit container including a plurality of at-home medical diagnostic test kits. In particular, the medical diagnostic test kit container may include a machine-readable code (e.g., QR code) configured to facilitate an augmented reality experience associated with the at-home medical diagnostic test kit container. In some embodiments, a testing and diagnostic platform may facilitate tracking of inventory within the at-home medical diagnostic test kit container and/or may facilitate coordination of prescription medication order fulfillment and delivery upon indication of a medical condition that could benefit from taking prescription medication.

TW 201033842 A describes a method of providing video medical information. The method comprises the steps of: (1) establish a medical information database, including a patient medical record and a video medical information; (2) provide an identity identification element having identity information that can be used for confirming the of patient's identity; (3) provide an identification device for identifying the identity identification element so as to read the identity information; (4) provide an electronic device to receive and confirm the identity information transmitted from the identification device, and read the medical record of the patient from the medical information database and playback the video medical information of the medical record. The video medical information displayed by this method is selected from the group composed of movies, texts, pictures, and sound, and is provided for the patient. The content of the video medical information is selected from the group composed of the patient data, medicine description, usage instructions and explanation of interaction of medicines, diet precaution during medication use and after cured, daily life precautions during medication use, and the information of medical visiting and diagnosis.

U.S. Publication No. 2022/0223283 A1 describes a system for electronic patient care including a gateway and a medical device. The gateway is configured to provide at least one of a routing functionality, a medical device software update, and a web service. The medical device is configured to operatively communicate with the gateway using the web service.

In other medical fields, methods for identification of medical staff is also known. For example, CN 103942850 A describes a medical staff on-duty monitoring method and a medical staff on-duty monitoring system based on video analysis and RFID (radio frequency identification) technology. The method comprises the steps of acquiring a video image in real time in a monitoring region; performing costume detection on a monitored video to acquire a medical staff detection result; selecting to perform face identification or walk identification according to the size of a face of the detected medical staff; performing motion detection to acquire on-duty information of the medical staff; detecting off-duty staff according to the video, and starting an RFID scanning program to determine a staff on-duty situation; furthermore, regularly starting an on-duty scanning program through RFID to judge on-duty situation of the medical staff, wherein if a suspected off-duty situation exists, the system requests a video monitoring system to confirm the situation; if off-duty information is determined, enabling the system to send alarming information of the off-duty situation of the medical staff.

CN 109426846 A describes a medical RFID recording bust tag including high-definition audio and video modules, a power supply unit, a lens assembly, a storage assembly and a medical personnel personal information card. The medical personnel personal information card is used for storing personal identity information of the medical personnel, and the lens assembly is used for recording audio and video information in the diagnosis and treatment process, and after being processed by the high-definition audio and video module, the audio and video information is recorded in the storage component for accessing and using.

In other technical fields, personal identification by using RFID technology and/or by visual inspection is also known, e.g., in DE 10 2016 103 694 A1, CN 206907120 U, BR 102014005719-6 A2, U.S. Publication No. 2006/0208066 A1, KR 20210027258 A or DE 10 2015 108 346 A1.

U.S. Publication No. 2014/0067421 A1 discloses an analyte measurement system including one or more handheld analyte meters and/or measurement devices and a means for collecting data, preserving data integrity, and uniquely identifying patient data received from multiple sources. For example, provided is a means to uniquely identify patients and their data when the data is collected from one or more measurement devices.

U.S. Publication No. 2022/0270720 A1 describes a method for adjusting a measurement setup used in an analytical method. The method includes a plurality of users carrying out analyte measurements that involve capturing images of an optical test strip having a test field and obtaining training data. The training data is then used to identify similarities and to identify a plurality of user profiles according to the similarities. Then, profile-specific measurement setup adjustments for at least one of the user profiles are provided for the individual user profile. An analytical method, an adjustment system and a mobile device are also disclosed.

U.S. Publication No. 2019/0012479 A1 discloses systems, devices, and methods for controlling access to a medical device. One example system includes an access card configured to wirelessly transmit user identification information and a medical device configured to receive the user identification information from the access card, and enable access to one or more components of the medical device based on permission information associated with the received user identification information. One example medical device includes a plurality of device components; a data receiver configured to wirelessly receive user identification information; a memory configured to store permission information in association with the user identification information; and a processor configured to retrieve the permission information from the memory in response to receiving the user identification information from the data receiver, and enable access to the one or more device components based on the retrieved permission information.

U.S. Publication No. 2019/0205578 A1 describes an infusion pump system including an infusion pump device and an RFID reader. The infusion pump device comprises at least a controller, a communication interface, and a fluidic pump. The RFID reader includes at least a microcontroller, a memory storage, an RFID manager, and an RFID reader communication device. The RFID manager is configured to read a user identification parameter from an RFID device. The RFID reader communication device of the RFID reader is configured to communicate with an access unit connected to a network, to query user-related information based on the user identification from a server over the network. The RFID reader communication device is configured to communicate with the communication interface of the infusion pump to forward the user related information to the infusion pump device.

Despite the advantages achieved by known methods or devices, several technical challenges remain. Specifically, user identification for mobile device-based medical and/or diagnostic applications may still be performed by human invigilators. However, the requirement for human invigilators to check the user's identity generally causes costs and restricts the application to operating hours of the identification service. Furthermore, this conventional identification method via camera recording of an ID card generally requires good ambient lighting situations. Additionally, visual assessment by human invigilators is rather cumbersome and should be avoided by, e.g., a more automated identification procedure.

### SUMMARY

The present disclosure teaches methods and devices which at least partially address the above-mentioned technical challenges. Specifically, methods and devices are disclosed which allow easy, reliable and convenient automatic identification of a user for mobile device-based analytical methods.

As used in the following, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B,” “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

Further, it shall be noted that the terms “at least one,” “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once. It shall also be understood for purposes of this disclosure and appended claims that, regardless of whether the phrases “one or more” or “at least one” precede an element or feature appearing in this disclosure or claims, such element or feature shall not receive a singular interpretation unless it is made explicit herein. By way of non-limiting example, the terms “analyte,” “camera,” “item of information” and “RFID identifier,” to name just a few, should be interpreted wherever they appear in this disclosure and claims to mean “at least one” or “one or more” regardless of whether they are introduced with the expressions “at least one” or “one or more.” All other terms used herein should be similarly interpreted unless it is made explicit that a singular interpretation is intended.

Further, as used in the following, the terms “preferably,” “more preferably,” “particularly,” “more particularly,” “specifically,” “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of this disclosure, a computer-implemented identification method of identifying a user for an analytical method of detecting at least one analyte in a sample of a body fluid is disclosed.

The term “computer-implemented” as used herein is a broad term and is to be given its ordinary and customary meaning to a person of ordinary skill in the art and is not to be limited to a special or customized meaning. The term specifically may refer, without limitation, to a method involving at least one computer and/or at least one computer network. The computer and/or computer network may comprise at least one processor which is configured for performing at least one of the method steps of the method according to this disclosure, such as of the identification method and/or of the analytical method, as will be outlined in further detail below. Preferably, each of the method steps may be performed by the computer and/or computer network. The computer-implemented method may be performed completely automatically, specifically without user interaction.

Referring to the computer-implemented aspects of the identification method, one or more of the method steps or even all of the method steps of the identification method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual analytical measurement.

The term “identifying” as used herein is a broad term and is to be given its ordinary and customary meaning to a person of ordinary skill in the art and is not to be limited to a special or customized meaning. The term specifically may refer, without limitation, to a process of determining an identity of a person, such as an identity of a user. The identity of a person may specifically comprise at least one characteristic item of information on the person, specifically at least one item of information distinguishing the person from one or more, specifically from all other, persons. The item of information may comprise one or more of a personal item of information, such as a name, an age, a gender, a date of birth and/or an address of the person, and a technically generated item of information, such as a number, a sequence of numbers, a sequence of alphanumeric text or other symbols associated with a person, e.g., in a specific application or environment. Consequently, the term “identification method” as used herein is a broad term and is to be given its ordinary and customary meaning to a person of ordinary skill in the art and is not to be limited to a special or customized meaning. The term specifically may refer, without limitation, to a process of identifying a user, i.e., of determining an identity of the user. The result of the identification method may comprise at least one characteristic item of information on the person, specifically at least one item of information distinguishing the person from one or more, specifically from all other, persons.

The term “user” as used herein is a broad term and is to be given its ordinary and customary meaning to a person of ordinary skill in the art and is not to be limited to a special or customized meaning. The term specifically may refer, without limitation, to a person, i.e., a human being, handling the mobile device. The user may specifically refer to a person independent from the fact that the human being may be in a healthy condition or may suffer from one or more diseases, such as a metabolic disease, e.g., diabetes, or a viral disease, e.g., a viral disease caused by an Influenza virus or SARS-CoV-2. However, additionally or alternatively, this disclosure may be applied to other types of users, patients or diseases.

The term “for an analytical method” as used herein is a broad term and is to be given its ordinary and customary meaning to a person of ordinary skill in the art and is not to be limited to a special or customized meaning. The term specifically may refer, without limitation, to the fact that the results of the identification method are configured for being used in the analytical method.

The term “analytical method” as used herein is a broad term and is to be given its ordinary and customary meaning to a person of ordinary skill in the art and is not to be limited to a special or customized meaning. The term specifically may refer, without limitation, to a quantitative and/or qualitative determination of the at least one property of the sample of the body fluid, such as at least one of a physical, a chemical and a biological property. The analytical method specifically may be an in vitro analytical method. The determination of the at least one property may specifically comprise quantitatively or qualitatively detecting at least one analyte in the sample of the body fluid. The result of the analytical method, as an example, may be at least one item of information on the at least one property of the sample, such as at least one item of information on the presence or absence of an analyte of interest. Alternatively or additionally, the result of the analytical method may comprise at least one item of information indicating a concentration of the at least one analyte of interest. The analyte may be or may comprise at least one arbitrary, dedicated and/or predetermined chemical or biological substance or species, such as at least one molecule or at least one chemical and/or biological compound. For example, the analyte may be or may comprise at least one specific virus and/or any parts thereof. The result of the analytical method may be or may comprise at least one item of information indicating the presence or absence of the virus or parts thereof in the sample of the body fluid. For example, the analyte may be a chemical compound which takes part in metabolism, such as one or more of glucose, lactate, cholesterol or triglycerides. For example, the analytical method may be a blood glucose measurement and, thus, the result of the analytical method may be a blood glucose concentration. Additionally or alternatively, the analytical method may relate to a method for detecting an RNA virus in a sample of body fluid. The method specifically may comprise the steps of releasing viral RNA from the sample, amplifying at least parts of the viral RNA comprised in said sample, and contacting the amplified viral RNA with at least one detecting reagent, such as at least one detecting reagent containing at least one nuclease, such as in lateral flow tests known in the art. As an example, the analytical method may comprise binding a viral antigen to an antibody, which may comprise a detectable label, such as a gold particle, a dye or the like. As another example, the analytical method may comprise performing a lateral flow test; appropriate methods are known in the art. Additionally or alternatively, other types of analytes or parameters may be determined, such as a pH value or the like. As will be understood by the skilled person, a “presence” of an analyte, for example, may be a presence of said analyte in an amount above a detection limit of the test used.

The term “detecting an analyte in a sample” as used herein is a broad term and is to be given its ordinary and customary meaning to a person of ordinary skill in the art and is not to be limited to a special or customized meaning. The term specifically may refer, without limitation, to a quantitative and/or qualitative determination of at least one analyte in an arbitrary sample. For example, the sample may comprise a body fluid, such as blood, interstitial fluid, urine, saliva or other types of body fluids. The result of the analytical method, as an example, may be a concentration of the analyte and/or the presence or absence of the analyte to be determined. Specifically, as an example, the analytical method may be a blood glucose measurement, thus the result of the analytical method may, for example, be a blood glucose concentration. In particular, an analytical measurement result value may be determined by the analytical method. Alternatively or additionally, the analytical method may comprise a lateral flow test for detecting a presence of at least one virus in the sample of the body fluid, such as a presence one or more of SARS-CoV-2, Influenza virus A and Influenza virus B.

The term “in vitro” as used herein is a broad term and is to be given its ordinary and customary meaning to a person of ordinary skill in the art and is not to be limited to a special or customized meaning. The term specifically may refer, without limitation, to a method which uses components of an organism and which is at least partially performed isolated from usual biological surroundings of the components of the organism. For example, the in vitro analytical method may comprise using the sample of the body fluid isolated from a user or patient, specifically using the sample of the body fluid in at least one optical test element, as will be outlined in further detail below.

The term “sample of a body fluid” as used herein is a broad term and is to be given its ordinary and customary meaning to a person of ordinary skill in the art and is not to be limited to a special or customized meaning. The term specifically may refer, without limitation, to an arbitrary aliquot part or aliquant part of a biological fluid which directly is a body fluid or which is derived from a body fluid, such as by one or more pre-processing steps, e.g., by transferring a body fluid to at least one sampling fluid, by diluting a body fluid, by centrifugation a body fluid or the like. The body fluid may comprise one or more of saliva, blood, interstitial fluid, urine or other types of body fluids. The sample of the body fluid may be collected via at least one nasopharyngeal swab, at least one swab of the anterior nares or from saliva, such by applying a cotton swab to a surface of the anterior nares and/or the throat. The collected sample of body fluid may be transferred to at least sampling or reagent fluid by immersing the cotton swab in the sampling or reagent fluid. The sampling or reagent fluid may specifically comprise lysis reagents. Alternatively or additionally, the sample of the body fluid may be a droplet of a body fluid as gathered from the body of a person, such as a droplet of saliva and/or blood and/or interstitial fluid or the like. The sample of the body fluid may specifically comprise at least one preparation of the body fluid, such as a cell preparation of the body fluid, e.g., a stained cell preparation of the body fluid. The sample of body fluid may also be simply referred to as the sample or the test sample.

The identification method comprises using a mobile device, specifically a mobile communication device, the mobile device having at least one camera and at least one RFID reader device.

The term “mobile device” as used herein is a broad term and is to be given its ordinary and customary meaning to a person of ordinary skill in the art and is not to be limited to a special or customized meaning. The term specifically may refer, without limitation, to a mobile electronics device, more specifically to a mobile communication device, such as a cell phone and/or a smartphone. Additionally or alternatively, the mobile device may also refer to a notebook, a tablet computer or another type of portable computer having at least one camera. Thus, generally, the mobile devices may be selected from the group consisting of: a cell phone having at least one camera, specifically a smartphone; a portable computer having at least one camera, specifically at least one of a notebook and a tablet computer. The mobile device may specifically be used both for the identification method and for the analytical method, as will be outlined in further detail below.

The term “camera” as used herein is a broad term and is to be given its ordinary and customary meaning to a person of ordinary skill in the art and is not to be limited to a special or customized meaning. The term specifically may refer, without limitation, to a device having at least one imaging element configured for recording or capturing spatially resolved one-dimensional, two-dimensional or even three-dimensional optical data or information. As an example, the camera may comprise at least one camera chip, such as at least one CCD chip and/or at least one CMOS chip configured for recording images. As used herein, without limitation, the term “image” specifically may relate to data recorded by using the camera, such as a plurality of electronic readings from the imaging device, such as the pixels of the camera chip.

The camera, besides the at least one camera chip or imaging chip, may comprise further elements, such as one or more optical elements, e.g., one or more lenses. As an example, the camera may be a fix-focus camera, having at least one lens which is fixedly adjusted with respect to the camera. Alternatively, however, the camera may also comprise one or more variable lenses which may be adjusted, automatically or manually. This disclosure specifically shall be applicable to cameras as usually used in mobile applications, such as notebook, computers, tablets or, specifically, cell phones such as smartphones. Thus, specifically, the camera may be part of the mobile device which, besides the at least one camera, comprises one or more data processing devices such as one or more data processors. Other cameras, however, are feasible.

The camera specifically may be a color camera. Thus, such as for each pixel, color information may be provided or generated, such as color values for three colors R, G, B. A larger number of color values is also feasible, such as four colors for each pixel, for example, R, G, G, B. Color cameras are generally known to the skilled person. Thus, as an example, each pixel of the camera chip may have three or more different color sensors, such as color recording pixels like one pixel for red (R), one pixel for green (G) and one pixel for blue (B). For each of the pixels, such as for R, G, B, values may be recorded by the pixels, such as digital values in the range of 0 to 255, depending on the intensity of the respective color. Instead of using color triples such as R, G, B, as an example, quadruples may be used, such as R, G, G, B or C, M, Y, K or the like. The color sensitivities of the pixels may be generated by color filters or by appropriate intrinsic sensitivities of the sensor elements used in the camera pixels. These techniques are generally known to the skilled person.

The mobile device may further comprise at least one processor. The term “processor” as used herein is a broad term and is to be given its ordinary and customary meaning to a person of ordinary skill in the art and is not to be limited to a special or customized meaning. The term specifically may refer, without limitation, to an arbitrary logic circuitry configured for performing basic operations of a computer or system, and/or, generally, to a device which is configured for performing calculations or logic operations. In particular, the processor may be configured for processing basic instructions that drive the computer or system. As an example, the processor may comprise at least one arithmetic logic unit (ALU), at least one floating-point unit (FPU), such as a math co-processor or a numeric co-processor, a plurality of registers, specifically registers configured for supplying operands to the ALU and storing results of operations, and a memory, such as an L1 and L2 cache memory. In particular, the processor may be a multi-core processor. Specifically, the processor may be or may comprise a central processing unit (CPU). Additionally or alternatively, the processor may be or may comprise a microprocessor, thus specifically the processor's elements may be contained in one single integrated circuitry (IC) chip. Additionally or alternatively, the processor may be or may comprise one or more application-specific integrated circuits (ASICs) and/or one or more field-programmable gate arrays (FPGAs) and/or one or more tensor processing unit (TPU) and/or one or more chip, such as a dedicated machine learning optimized chip, or the like. The processor specifically may be configured, such as by software programming, for performing one or more evaluation operations as will be outlined in further detail below.

The term “RFID” as used herein is a broad term and is to be given its ordinary and customary meaning to a person of ordinary skill in the art and is not to be limited to a special or customized meaning. The term specifically may refer, without limitation, to radio-frequency electromagnetic fields used for data exchange, such as for identification purposes. RFID techniques are known to the skilled person, e.g., according to one or more of the ISO/IEC 14443 standard or the ISO/IEC 18000 standard, in particular according to one or more of the respective standard in a valid version at the date of this document.

The term “RFID reader device” as used herein is a broad term and is to be given its ordinary and customary meaning to a person of ordinary skill in the art and is not to be limited to a special or customized meaning. The term specifically may refer, without limitation, to a device configured for RFID. Specifically, the RFID reader device may be configured to read out an RFID tag or chip by using radio-frequency electromagnetic radiation. The RFID reader device may be configured to read out data stored by an RFID tag or chip by decoding the radio-frequency signal. The RFID reader device, as an example, may comprise a near-filed communication (NFC) standard communication interface or NFC reader.

The identification method comprises the following method steps, which specifically may be performed in the given order. However, a different order may also be possible. It is further possible to perform one, more than one or even all of the method steps repeatedly. Further, it is possible to perform two or more of the method steps in a fashion overlapping in time or fully or partially simultaneously.

The identification method comprises:
a. prompting the user to identify by bringing at least one RFID identifier of the user into a communication range of the RFID reader device;
b. reading out data from the RFID identifier by using the RFID reader device;
c. validating the data read out in step b., wherein the validating comprises checking if at least one valid item of identification information of the user was successfully transferred with the data from the RFID identifier to the RFID reader device.

The term “prompting” as used herein is a broad term and is to be given its ordinary and customary meaning to a person of ordinary skill in the art and is not to be limited to a special or customized meaning. The term specifically may refer, without limitation, to at least one of requesting, asking, suggesting and inviting the user to perform a specific action. Specifically, the prompting may comprise providing a notification to the user, such as one or more of a visual notification, an acoustic notification and a haptic notification, the notification specifically requesting the user to perform the specific action.

The term “RFID identifier” as used herein is a broad term and is to be given its ordinary and customary meaning to a person of ordinary skill in the art and is not to be limited to a special or customized meaning. The term specifically may refer, without limitation, to an arbitrary element or device configured for storing RFID-readable data. The RFID identifier may specifically comprise at least one RFID tag or chip, such as a label configured for exchanging the stored data with the RFID reader device by using radio-frequency electromagnetic radiation, e.g., by using the NFC standard. The RFID tag or chip may comprise an antenna configured to receive and to emit a radio-frequency signal and an electronic chip, such as a microchip, configured to store the data. Specifically, the RFID tag may be a flexible substrate having an electronic conducting coil and optionally at least one microchip. The data stored on the RFID identifier may specifically comprise at least one characteristic item of information on the user, specifically at least the item of identification information on the user. For example, the RFID identifier may be an identification card, e.g., an official identification card, comprising one or more RFID tags or chips. For example, the RFID identifier may be an electronic identification card, e.g., the German identity card (“Personalausweis”) or the electronic identification card (“eID-card”) for citizen from the EU and the EEA. Other examples are also feasible.

The RFID identifier may comprise a 13.56 MHz RFID chip, specifically a 13.56 MHz RFID chip using the ISO 7816 protocols. The RFID identifier may be compatible with at least one of standards: ISO 18000-3; ISO 14443, specifically ISO/IEC 14443-4:2018.

The term “communication range” as used herein is a broad term and is to be given its ordinary and customary meaning to a person of ordinary skill in the art and is not to be limited to a special or customized meaning. The term specifically may refer, without limitation, to at least one interval of distance between the RFID reader device and the RFID identifier in which data transfer between the RFID reader device and the RFID identifier is enabled. Specifically, the communication range may comprise at least one class or set of admissible values of a distance between the RFID reader device and the RFID identifier. Having the RFID reader device and the RFID identifier distanced from each other with a distance in the class or set of admissible values, data may be transferred between the RFID reader device and the RFID identifier. The communication range may specifically comprise a narrow interval of allowed distances, for example, a distance of 4 cm or less, and, thus, specifically, rendering the data transfer between the RFID reader device and the RFID identifier a near-field communication (NFC).

The term “reading out” as used herein is a broad term and is to be given its ordinary and customary meaning to a person of ordinary skill in the art and is not to be limited to a special or customized meaning. The term specifically may refer, without limitation, to process of retrieving data, such as data stored in the RFID identifier, in an electronic format. The reading out may specifically comprise an electronic reading out, such as a reading out by near field communication (NFC). The reading out of data from the RFID identifier may specifically comprise one or more of querying data, transferring data and/or obtaining data from the RFID identifier, specifically via the radio-frequency signal of the RFID reader device and/or RFID identifier.

The term “data” as used herein is a broad term and is to be given its ordinary and customary meaning to a person of ordinary skill in the art and is not to be limited to a special or customized meaning. The term specifically may refer, without limitation, to at least one numerical and/or alphanumerical item of information, specifically conveying information on an identity of the user. Specifically, the data read out from the RFID identifier may comprise at least one a personal item of information, such as a name, an age, a gender, a date of birth and/or an address of the person, and a technically generated item of information, such as a number, a sequence of numbers, a sequence of alphanumeric text or other symbols associated with a person, e.g., in a specific application or environment.

The readout in step b. may comprise a secure data transmission of the at least one item of identification information from the RFID identifier to the RFID reader device. The secure data transmission may comprise the use of at least one of an error detection and an error correction. The error detection may specifically comprise detecting errors in the data transferred from the RFID identifier to the RFID reader device, e.g., errors caused by noise or other impairments during transmission from the RFID identifier to the RFID reader device. The error correction may specifically comprise correcting detected errors in the data transferred from the RFID identifier to the RFID reader device, e.g., correcting errors in the data transferred from the RFID identifier to the RFID reader device in order to reconstruct at least one valid item of identification information. Such error detection methods are known to the skilled person. Alternatively or additionally, the readout in step b. may comprise a transmission of the at least one item of identification information from the RFID identifier to the RFID reader device in an encoded format. The encoded format may comprise the use of a Hamming code.

Data made available by the RFID identifier of the user for read out by the RFID reader device may comprise at least the item of identification information and at least one of an error detection portion and an error correction portion. The validating in step c. may comprise evaluating the error detection portion or error correction portion, respectively.

The term “validating” as used herein is a broad term and is to be given its ordinary and customary meaning to a person of ordinary skill in the art and is not to be limited to a special or customized meaning. The term specifically may refer, without limitation, to a process of evaluating at least one state of data. Specifically, the validating may comprise evaluating one or more of a qualitative state and a quantitative state of data. The validating may comprise ensuring that the state of data meets given or pre-defined requirements, e.g., ensuring that the data are complete, in a correct and/or useful state. For example, the validating may comprise evaluating one or more of a content and a format of data in order to ensure that the data are complete and correct and/or useful for further processing. The validating in step c. may specifically be affirmative in case at least one valid item of identification information was successfully transferred with the data from the RFID reader device. A result of step c., specifically, a result of the validating, may comprise the valid item of identification information, specifically at least one valid item of identification information read out from the RFID identifier.

The term “item of identification information” as used herein is a broad term and is to be given its ordinary and customary meaning to a person of ordinary skill in the art and is not to be limited to a special or customized meaning. The term specifically may refer, without limitation, to at least one item of information, e.g., at least one numerical and/or alphanumerical item of information, configured for identifying a person. The item of identification information may be characteristic for a person, specifically for the user. The person or user may be distinguished from other persons or users by means of the item of identification information. The item of identification information may comprise one or more of a personal item of information, such as a name, an age, a gender, a date of birth and/or an address of the person, and a technically generated item of information, such as a number, a sequence of numbers, a sequence of alphanumeric text or other symbols associated with a person, e.g., in a specific application or environment.

The term “valid” as used herein, specifically in the context of a “valid item of information,” is a broad term and is to be given its ordinary and customary meaning to a person of ordinary skill in the art and is not to be limited to a special or customized meaning. The term specifically may refer, without limitation, to the fact that an item of information is correct in terms of a content of the item of information. Specifically, the valid item of identification information may be or may comprise at least one correct item of identification information in terms of its content, such as in terms of correct information conveyed by the item of identification information, e.g., with respect to a personal item of information and/or a technically generated item of information, as outlined above. In other words, the fact of having a valid item of identification information may refer to the fact that data being validated in step c. comprises at least one required, necessary and/or expected item of identification information, such as at least a name, an age and/or a gender of the user.

The term “successful” as used herein, specifically in the context of a “successful transfer,” is a broad term and is to be given its ordinary and customary meaning to a person of ordinary skill in the art and is not to be limited to a special or customized meaning. The term specifically may refer, without limitation, to the fact that an item of information is complete in terms of a content of the item of information. Specifically, the successfully transferred item of identification information may be or may comprise at least one completely transferred item of information in terms of its content, such as in terms of information conveyed by the item of identification information, e.g., with respect to a personal item of information and/or a technically generated item of information, as outlined above. The successfully transferred item of identification information may specifically comprise any item of information required for identifying the user, e.g., at least a name, an age and/or a gender of the user.

In step c., the checking of the data for at least one valid item of identification information on the user transferred with the data from the RFID identifier to the RFID reader device comprises at least one of:
checking if the at least one item of identification information on the user was successfully and completely transferred with the data from the RFID identifier to the RFID reader device;
checking if the at least one item of identification information on the user transferred with the data from the RFID identifier to the RFID reader device is sufficient, specifically is transferred within an expiry time span, more specifically an expiry time span defined in the data transferred from the RFID identifier to the RFID reader device;
checking if the user is authorized to perform the analytical method, specifically if the user's age fulfills at least one predetermined age condition;
checking if the at least one item of identification information on the user matches at least one previously registered item of identification information on the user, e.g., checking if the at least one item of identification information on the user matches at least one item of identification information on the user previously registered in a database, such as a database on the mobile device and/or a cloud-based database;
checking if the at least one item of identification information on the user was transferred from the RFID identifier to the RFID reader device within at least one admissible region, e.g., having an address of the user and/or location data of the mobile device in an admissible region.

Alternatively or additionally, step c. may comprise determining at least one checksum of the data transferred from the RFID identifier to the RFID reader device. The checksum of the data may be used for checking if the data transferred from the RFID identifier to the RFID reader device comprises the at least one valid item of identification information on the user being successfully transferred with the data from the RFID identifier to the RFID reader device.

In case at least one valid item of identification information of the user has been successfully transferred with the data from the RFID identifier to the RFID reader device, step c. may comprise generating a user ID profile for the user, specifically on the mobile device. The term “user ID profile” as used herein is a broad term and is to be given its ordinary and customary meaning to a person of ordinary skill in the art and is not to be limited to a special or customized meaning. The term specifically may refer, without limitation, to a collection of data related to a user. Specifically, the user ID profile may comprise user-specific data. The user ID profile may specifically comprise the at least one valid item of identification information. Additionally or alternatively, the user ID profile may comprise at least one item of ID profile generation information on the user, the at least one item of ID profile generation information comprising information on the result of step c.. For example, the item of ID profile generation information may comprise at least one item of information indicating that the valid item of identification information of the user was successfully transferred. Additionally or alternatively, the user ID profile may comprise user-specific operational data, e.g., settings of an application running of the mobile device, specifically allowing performing the analytical method adjusted to the user's habits or capabilities.

In case no valid item of identification information of the user has been successfully transferred with the data from the RFID identifier to the RFID reader device, step c. may comprise generating no user ID profile for the user. Alternatively or additionally, in case no valid item of identification information of the user was successfully transferred with the data from the RFID identifier to the RFID reader device, the identification method may further comprise prompting the user at least one item of failure information. The item of failure information may indicate at least one of a read out of an invalid item of identification information and a failed transfer of the item of identification information. The user may be prompted to repeat performing steps a. to c..

Step a. may further comprise prompting the user to enter a personal identification number (PIN), specifically a six-digit decimal access code, into the mobile device, specifically via at least one user interface of the mobile device, more specifically via at least one keypad.

In a further aspect of this disclosure, a computer-implemented analytical method for detecting at least one analyte in a sample of a body fluid of a user is disclosed. For definitions of terms and/or possible embodiments used in the analytical method, reference is made to the description of the identification method above.

The analytical method comprises using a mobile device, specifically a mobile communication device, the mobile device having at least one camera and at least one RFID reader device.

The analytical method comprises the following method steps, which specifically may be performed in the given order. However, a different order may also be possible. It is further possible to perform one, more than one or even all of the method steps repeatedly. Further, it is possible to perform two or more of the method steps in a fashion overlapping in time or fully or partially simultaneously.

The analytical method comprises:
i. evaluating if a validating step c. of a previously performed identification method according to this disclosure, such as according to any one of the embodiments disclosed above and/or according to any one of the embodiments disclosed in further detail below, was successful, indicating that at least one valid item of identification information of the user has been successfully transferred with the data from the RFID identifier to the RFID reader device;
ii. depending on the outcome of step i., taking one of the following actions:
   ii.1. if the evaluation in step i. is affirmative, specifically indicating that at least one valid item of identification information of the user has been successfully transferred from the user's RFID identifier to the RFID reader device of the user's mobile device, initiating at least one analytical step, the at least one analytical step comprising detecting the analyte by evaluating at least one image of at least one detection portion of at least one test element having the sample of the body fluid of the user applied thereto, the at least one image being acquired with the camera;
   ii.2. if the evaluation in step i. is negative, specifically indicating that no valid item of identification information of the user has been successfully transferred from the user's RFID identifier to the RFID reader device of the user's mobile device, initiating at least one action selected from the group consisting of:
      performing the identification method according to this disclosure, such as according to any one of the embodiments disclosed above and/or according to any one of the embodiments disclosed in further detail below;
      generating at least one item of preventing information, the item of preventing information indicating that the analytical step is to be prevented.

The term “evaluating,” also referred to as “evaluation,” as used herein is a broad term and is to be given its ordinary and customary meaning to a person of ordinary skill in the art and is not to be limited to a special or customized meaning. The term specifically may refer, without limitation, to a process of processing data and deriving at least one representative result therefrom. The evaluating may comprise processing the data transferred from the RFID identifier to the RFID reader device, specifically processing the successfully transferred valid item of identification information. Alternatively or additionally, the evaluating may comprise processing data from a user ID profile, specifically a user ID profile being generated in the previously performed identification method. The evaluating may comprise deriving at least one item of confirmation information on a presence of the successfully transferred valid item of identification information, e.g., a Boolean, such as “true” or “yes” in case the at least one valid item of identification information of the user has been successfully transferred with the data from the RFID identifier to the RFID reader device and “false” or “no” in case the at least one valid item of identification information of the user has not been successfully transferred with the data from the RFID identifier to the RFID reader device or no valid item of identification information has been transferred with the data from the RFID identifier to the RFID reader device.

The term “previously” as used herein, specifically in the context of the identification method, is a broad term and is to be given its ordinary and customary meaning to a person of ordinary skill in the art and is not to be limited to a special or customized meaning. The term specifically may refer, without limitation, to the fact the identification method is performed prior to the analytical method. Thus, the identification method may be performed at least once before the analytical method.

The evaluating in step i. may comprise checking for available user ID profiles, specifically user ID profiles generated by the previously performed identification method. In case two or more user ID profiles are available, specifically two or more user ID profiles for different users, the analytical method may comprise prompting the user to select a user ID profile or to confirm a pre-selected user ID profile, specifically a pre-selected user ID profile corresponding to the user ID profile from a last login. The item of identification information of the user may be retrieved from the available user ID profile, specifically from the selected user ID profile. Additionally, the evaluating in step i. may comprise prompting the user to confirm validity of the retrieved item of identification information. In case the user confirms validity of the retrieved item of identification information, the evaluation in step i. may be affirmative. In case the user denies validity of the retrieved item of identification information, the evaluation in step i. may be negative.

The term “affirmative” as used herein is a broad term and is to be given its ordinary and customary meaning to a person of ordinary skill in the art and is not to be limited to a special or customized meaning. The term specifically may refer, without limitation, to a positive outcome of the evaluating in step i.. Specifically, the evaluation in step i. may be affirmative in case the at least one valid item of identification information of the user has been successfully transferred with the data from the RFID identifier to the RFID reader device.

The term “analytical step” as used herein is a broad term and is to be given its ordinary and customary meaning to a person of ordinary skill in the art and is not to be limited to a special or customized meaning. The term specifically may refer, without limitation, to a process of generating data and evaluating data in the analytical method, as defined above. Thus, the analytical step may comprise detecting the at least one analyte in the sample of the body fluid. The analytical step may comprise generating image data, e.g., by capturing at least one image of the test element having the sample of the body fluid applied thereto and/or by retrieving image data, and evaluating the image data to obtain information on a concentration and/or a presence of the analyte in the sample of the body fluid. The analytical step may comprise processing the image data, e.g., one or more of a section of the image and/or at least one color value of the image, and deriving a concentration and/or a presence of the analyte in the sample of the body fluid therefrom. The analytical step may, as an example, comprise using at least one calibration function for processing the image data, the calibration function linking the image data, e.g., the color value derived from the image, to the concentration of the analyte in the sample of the body fluid therefrom.

The at least one analytical step may further comprise, specifically prior to the evaluating of the at least one image, performing at least one of the following actions:
prompting the user to apply the sample of the body fluid to the at least one test element;
prompting the user to confirm application of the sample of the body fluid to the at least one test element;
displaying at least one waiting time between sample application and capturing of the at least one image, e.g., for one or more seconds, specifically for one or more minutes, which may specifically be dependent on the test element, wherein during the waiting time, no image capturing may be allowed;
capturing the at least one image of the at least one detection portion of the test element having the sample of the body fluid of the user applied thereto using the camera of the mobile device;
retrieving the at least one image of the at least one detection portion of the test element having the sample of the body fluid of the user applied thereto.

The term “test element” as used herein is a broad term and is to be given its ordinary and customary meaning to a person of ordinary skill in the art and is not to be limited to a special or customized meaning. The term specifically may refer, without limitation, to an arbitrary element or device configured for performing an optical detection reaction, for example, a color-change detection reaction and/or a reaction during which one or more optically detectable features on or within the test element become visible, such as one or more markings, such as linear markings known from rapid COVID testing. The test element may specifically be an optical test element. The test element may, as an example, be embodied as a test strip or as a test stick. The test element may particularly have the at least one test field containing at least one test chemical being sensitive for the property of the sample, such as for detecting the at least one analyte. The test element may, as an example, comprise one or more application sites for applying the at least one sample. The application site may be different from the position of the test field and may be fluidically connected to the test field, such as by one or more capillary elements, such as one or more porous elements capable of transporting liquid. The test element, as an example, may comprise at least one substrate, such as at least one carrier, with the at least one test field applied thereto or integrated therein. The test element may comprise at least one control area, specifically in a proximity to the test field, for example, enclosing or surrounding the test field and/or arranged behind the test field in a direction of flow of the sample on the test element. The control area may be a separate field independently arranged on the substrate or carrier. The control area may be configured for indicating correct applying of the sample of the body fluid on the test element. The carrier, as an example, may be strip-shaped, thereby rendering the test element a test strip. These test strips are generally widely in use and available. One test strip may carry a single test field or a plurality of test field having identical or different test chemicals comprised therein. Additionally or alternatively, the test element may be embodied as a stick or chip, e.g., with a housing having the above-mentioned substrate disposed therein, e.g., a housing having one or more application openings for applying the sample and one or more detection windows for enabling an optical detection of the at least one detection reaction. For example, the test element may be configured for performing at least one color-change detection reaction for detecting blood glucose in the sample of the body fluid, such as a blood glucose test. Alternatively or additionally, as another example, the test element may comprise at least one lateral flow assay, specifically at least one lateral flow assay configured for performing at least one reaction during which one or more optically detectable features on or within the test element become visible for detecting a presence of a virus in the sample of the body fluid, such as a rapid antigen test. Various further options may also be feasible.

The term “detection portion” as used herein is a broad term and is to be given its ordinary and customary meaning to a person of ordinary skill in the art and is not to be limited to a special or customized meaning. The term specifically may refer, without limitation, to a region of the test element having a coherent amount of the test chemical, such as to a field, e.g., a field of round, polygonal or rectangular shape, having one or more layers of material, with at least one layer of the detection portion having the test chemical comprised therein. Other layers may be present providing specific optical properties such as reflective properties, providing spreading properties for spreading the sample or providing separation properties such as for separating of particulate components of the sample, such as cellular components. The detection portion may be the test field of the test element, as described above. The sample of the body fluid may be applied directly to the detection portion, e.g., for blood glucose measurements where a droplet of blood may be directly applied to the test element comprising the detection portion, or may be applied indirectly to the detection portion, e.g., by applying the sample of the body fluid to a reservoir or an application site of the test element, wherein the sample of the body fluid may flow from the reservoir of application site to the detection portion of the test element, such as by capillary forces acting on the sample of the body fluid.

The test element may comprise at least one element selected from the group consisting of: a lateral flow assay, specifically a lateral flow assay for detecting a presence and/or a concentration of the analyte in the sample of the body fluid, more specifically a lateral flow assay for detecting a presence one or more of SARS-CoV-2, Influenza virus A and/or Influenza virus B in the sample of the body fluid; an optical test element, specifically an optical test element configured for changing at least one optically detectable property of at least one feature when the analyte is detected in the sample of the body fluid, more specifically an optical test element for changing at least one optically detectable property depending on a concentration of the analyte in the sample of the body fluid.

The term “negative” as used herein is a broad term and is to be given its ordinary and customary meaning to a person of ordinary skill in the art and is not to be limited to a special or customized meaning. The term specifically may refer, without limitation, to a rejecting outcome of the evaluating in step i.. Specifically, the evaluation in step i. may be negative in case the at least one valid item of identification information of the user has not been successfully transferred with the data from the RFID identifier to the RFID reader device or no valid item of identification information has been transferred with the data from the RFID identifier to the RFID reader device.

The term “item of preventing information” as used herein is a broad term and is to be given its ordinary and customary meaning to a person of ordinary skill in the art and is not to be limited to a special or customized meaning. The term specifically may refer, without limitation, to at least one item of information indicating at least one hindered action. The item of preventing information may comprise information to the user that the analytical step is to be prevented, specifically via visible, acoustic and/or haptic means, e.g., a notification display on a display of the mobile device.

The method may further comprise assigning an outcome of the analytical step to the valid item of identification information. Additionally or alternatively, the method may further comprise providing at least one certificate on an outcome of the analytical step, specifically a personalized certificate being issued for the identified user.

Alternatively or additionally, the at least one action initiated in step ii.2 may further comprise performing the analytical step for personal information only, specifically without providing a certificate on an outcome of the analytical step.

In a further aspect of this disclosure, a computer program is disclosed, comprising instructions which, when the program is executed by a mobile device having at least one camera and at least one RFID reader device cause the mobile device to perform at least one of the identification method and the analytical method according to this disclosure, such as according to any one of the embodiments disclosed above and/or according to any one of the embodiments disclosed in further detail below, respectively.

Similarly, a computer-readable storage medium, specifically a non-transient computer-readable storage medium, is disclosed, comprising instructions which, when the instructions are executed by a mobile device having at least one camera and at least one RFID reader device cause the mobile device to perform at least one of the identification method and the analytical method according to this disclosure, such as according to any one of the embodiments disclosed above and/or according to any one of the embodiments disclosed in further detail below, respectively.

As used herein, the term “computer-readable storage medium” specifically may refer to non-transitory data storage means, such as a hardware storage medium having stored thereon computer-executable instructions. The computer-readable storage medium specifically may be or may comprise a storage medium such as a random-access memory (RAM) and/or a read-only memory (ROM). The computer-readable storage medium may specifically comprise at least one computer-readable data carrier.

The methods and devices according to this disclosure may provide a larger number of advantages over known methods and devices of similar kind. Specifically, the identification method may allow avoiding cumbersome identification via camera recording of an ID card involving a human invigilators but instead may provide a smartphone-based, automated identification based on RFID for analytical methods performed using mobile devices. The identification method may improve user experience of mobile device-based analytical methods by a more convenient handling of an application running on the mobile device in cases where an official test certificate is needed.

Specifically, by using the methods according to this disclosure, the analytical method may be provided in an application running on the mobile device, in particular for home-testing, wherein images of the test element, e.g., a lateral flow test assay, such as rapid antigen tests, or blood glucose test element, may be captured by using the camera of the mobile device. From optically detectable changes within the test field, such as graphical elements like bars, or color formation, a presence of an analyte and/or an analyte concentration may be determined. For providing official test certificates, the user can be identified for the performed test, e.g., in cases where an official test certificate may be required. To this end, the identification method may provide an RFID-based identification method that can be used for mobile device-based diagnostics solutions. The methods may allow using any ID card, e.g., electronic ID cards comprising RFID chips like the German Personalausweis, the ID card being brought into close proximity to the mobile device to securely readout the identity of the user.

The analytical method may comprise, as an example, using lateral flow assays, particularly for Sars-CoV-2 home test setting. The identification method may allow generating user ID profiles storing personal ID profile information. The mobile device may be regarded as a rather personal device. The analytical method may check whether a digital or electronic user ID profile already exists, such as a user ID profile stored locally on the mobile device or in a cloud-based data storage. If this is the case, the user may be prompted to confirm his identity in order to exclude accidental activation, e.g., with a shared device like a family/kids phone. Optionally, multiple user ID profiles may be stored and one of the stored user ID profiles may be selected for use. In case of either no existing user ID profiles available or an application without the user ID storage option, the user may be prompted to identify via the identification method using the RFID technology of the mobile device. In case of a read out failure, the analytical method may comprise prevent the analytical step. Optionally, the analytical step may be allowed for personal information only, without providing a test certificate. The analytical method can be applied to any digital test based comprising use of mobile devices, particularly detecting an absence, a presence and/or a concentration of the analyte in the sample of the body fluid.

The identification method may provide an easy automatic identification of the user since modern ID cards, e.g., the German “Personalausweis” or electronic ID card for citizens of the EU and the EEA, provide a digital identification via an incorporated RFID chip. The RFID chip, also referred to as electronic ID, may store the item of identification information on the user. Most modern mobile device comprise RFID reader devices and, thus, provide capabilities to read out RFID chips in the ID card. Therefore, instead of rather cumbersome identification via camera recording of the ID card and visual assessment from a human invigilators, the ID card can be read out by means of RFID automatically to provide an automatic identification method. Optionally, the identification method may allow generating user ID profiles and the subsequent analytical method may allow selecting from the previously generated user ID profiles which may further improves ease of use.

As an example, the analytical method may be started by a user opening an application running on the mobile device. Optionally, the analytical method may comprise checking whether a user ID profile already exists, e.g., stored locally in the app on the mobile device or in an app-related cloud-based storage device. In case a user ID profile exists, the analytical method may comprise requesting the user to confirm his identity, specifically in order to exclude that by accident, a shared mobile device, e.g., a family or kids mobile device may lead to a wrong assignment of the test result. Further, the analytical method may comprise prompting the user to choose between multiple user ID profiles.

In case of either no existing user ID profile available or if the mobile device may not allow storing user ID profiles, the analytical method may request the user to identify by bringing the RFID identifier into the communication range of the RFID reader device, specifically by requesting bringing the RFID identifier in close proximity to the mobile device to securely readout the item of identification information. In case the valid item of identification information may be successfully read out, specifically having no error occurring during the transmission, e.g., being checked via checksums, analytical method may proceed with the actual analytical step. In all other cases, the analytical method may comprise preventing any analytical step or, optionally, allowing the analytical step for personal information only, e.g., without providing a test certificate. The identification method can be applied to any analytical method comprising a diagnostic app with person identification.

Summarizing and without excluding further possible embodiments, the following embodiments may be envisaged:
Embodiment 1: A computer-implemented identification method of identifying a user for an analytical method of detecting at least one analyte in a sample of a body fluid, the identification method comprising using a mobile device, specifically a mobile communication device, the mobile device having at least one camera and at least one RFID reader device, the identification method comprising:
a. prompting the user to identify by bringing at least one RFID identifier of the user into a communication range of the RFID reader device;
b. reading out data from the RFID identifier by using the RFID reader device;
c. validating the data read out in step b., wherein the validating comprises checking if at least one valid item of identification information of the user was successfully transferred with the data from the RFID identifier to the RFID reader device.
Embodiment 2: The identification method according to the preceding embodiment, wherein a result of step c. comprises the valid item of identification information.
Embodiment 3: The identification method according to any one of the preceding embodiments, wherein, in case at least one valid item of identification information of the user has been successfully transferred with the data from the RFID identifier to the RFID reader device; step c. comprises generating a user ID profile for the user, specifically on the mobile device.
Embodiment 4: The identification method according to the preceding embodiment, wherein the user ID profile comprises the at least one valid item of identification information.
Embodiment 5: The identification method according to any one of the two preceding embodiments, wherein the user ID profile comprises at least one item of ID profile generation information on the user, the at least one item of ID profile generation information comprising information on the result of step c.
Embodiment 6: The identification method according to any one of the preceding embodiments, wherein, in case no valid item of identification information of the user has been successfully transferred with the data from the RFID identifier to the RFID reader device, step c. comprises generating no user ID profile for the user.
Embodiment 7: The identification method according to any one of the preceding embodiments, wherein, in case no valid item of identification information of the user was successfully transferred with the data from the RFID identifier to the RFID reader device, the identification method further comprises prompting the user at least one item of failure information.
Embodiment 8: The identification method according to the preceding embodiment, wherein the item of failure information indicates at least one of a read out of an invalid item of identification information and a failed transfer of the item of identification information.
Embodiment 9: The identification method according to any one of the preceding embodiments, wherein, in step c., the checking of the data for at least one valid item of identification information on the user transferred with the data from the RFID identifier to the RFID reader device comprises at least one of:
checking if the at least one item of identification information on the user was successfully and completely transferred with the data from the RFID identifier to the RFID reader device;
checking if the at least one item of identification information on the user transferred with the data from the RFID identifier to the RFID reader device is sufficient, specifically is transferred within an expiry time span, more specifically an expiry time span defined in the data transferred from the RFID identifier to the RFID reader device;
checking if the user is authorized to perform the analytical method, specifically if the user's age fulfills at least one predetermined age condition;
checking if the at least one item of identification information on the user matches at least one previously registered item of identification information on the user;
checking if the at least one item of identification information on the user was transferred from the RFID identifier to the RFID reader device within at least one admissible region.
Embodiment 10: The identification method according to any one of the preceding embodiments, wherein the readout in step b. comprises a secure data transmission of the at least one item of identification information from the RFID identifier to the RFID reader device.
Embodiment 11: The identification method according to the preceding embodiment, wherein the secure data transmission comprises the use of at least one of an error detection and an error correction.
Embodiment 12: The identification method according to any one of the preceding embodiments, wherein the readout in step b. comprises a transmission of the at least one item of identification information from the RFID identifier to the RFID reader device in an encoded format.
Embodiment 13: The identification method according to the preceding embodiment, wherein the encoded format comprises the use of a Hamming code.
Embodiment 14: The identification method according to any one of the preceding embodiments, wherein the RFID identifier comprises a 13.56 MHz RFID chip, specifically a 13.56 MHz RFID chip using the ISO 7816 protocols.
Embodiment 15: The identification method according to any one of the preceding embodiments, wherein the RFID identifier is compatible with at least one of standards: ISO 18000-3; ISO 14443, specifically ISO/IEC 14443-4:2018.
Embodiment 16: The identification method according to any one of the preceding embodiments, wherein step a. further comprises prompting the user to enter a personal identification number (PIN), specifically a six-digit decimal access code, into the mobile device, specifically via at least one user interface of the mobile device, more specifically via at least one keypad.
Embodiment 17: The identification method according to any one of the preceding embodiments, wherein step c. comprises determining at least one checksum of the data transferred from the RFID identifier to the RFID reader device.
Embodiment 18: The identification method according any one of the preceding embodiments, wherein data made available by the RFID identifier of the user for read out by the RFID reader device comprises at least the item of identification information and at least one of an error detection portion and an error correction portion, wherein the validating in step c. comprises evaluating the error detection portion or error correction portion, respectively.
Embodiment 19: A computer-implemented analytical method for detecting at least one analyte in a sample of a body fluid of a user, the analytical method comprising using a mobile device, specifically a mobile communication device, the mobile device having at least one camera and at least one RFID reader device, the analytical method comprising:
i. evaluating if a validating step c. of a previously performed identification method according to any one of the preceding embodiments was successful, indicating that at least one valid item of identification information of the user has been successfully transferred with the data from the RFID identifier to the RFID reader device;
ii. depending on the outcome of step i., taking one of the following actions:
   ii.1. if the evaluation in step i. is affirmative, specifically indicating that at least one valid item of identification information of the user has been successfully transferred from the user's RFID identifier to the RFID reader device of the user's mobile device, initiating at least one analytical step, the at least one analytical step comprising detecting the analyte by evaluating at least one image of at least one detection portion of at least one test element having the sample of the body fluid of the user applied thereto, the at least one image being acquired with the camera;
   ii.2. if the evaluation in step i. is negative, specifically indicating that no valid item of identification information of the user has been successfully transferred from the user's RFID identifier to the RFID reader device of the user's mobile device, initiating at least one action selected from the group consisting of:
      performing the identification method according to any one of the preceding embodiments;
      generating at least one item of preventing information, the item of preventing information indicating that the analytical step is to be prevented.
Embodiment 20: The analytical method according to the preceding embodiment, wherein the test element comprises at least one element selected from the group consisting of: a lateral flow assay, specifically a lateral flow assay for detecting a presence and/or a concentration of the analyte in the sample of the body fluid, more specifically a lateral flow assay for detecting a presence one or more of SARS-CoV-2, Influenza virus A and/or Influenza virus B in the sample of the body fluid; an optical test element, specifically an optical test element configured for changing at least one optically detectable property of at least one feature when the analyte is detected in the sample of the body fluid, more specifically an optical test element for changing at least one optically detectable property depending on a concentration of the analyte in the sample of the body fluid.
Embodiment 21: The analytical method according to any one of the preceding embodiments referring to an analytical method, the at least one analytical step further comprising, specifically prior to the evaluating of the at least one image, performing at least one of the following actions:
prompting the user to apply the sample of the body fluid to the at least one test element;
prompting the user to confirm application of the sample of the body fluid to the at least one test element;
displaying at least one waiting time between sample application and capturing of the at least one image;
capturing the at least one image of the at least one detection portion of the test element having the sample of the body fluid of the user applied thereto using the camera of the mobile device;
retrieving the at least one image of the at least one detection portion of the test element having the sample of the body fluid of the user applied thereto.
Embodiment 22: The analytical method according to any one of the preceding embodiments referring to an analytical method, wherein the evaluating in step i. comprises checking for available user ID profiles, specifically user ID profiles generated by the previously performed identification method.
Embodiment 23: The analytical method according to the preceding embodiment, wherein, in case two or more user ID profiles are available, specifically two or more user ID profiles for different users, the analytical method comprises prompting the user to select a user ID profile or to confirm a pre-selected user ID profile, specifically a pre-selected user ID profile corresponding to the user ID profile from a last login.
Embodiment 24: The analytical method according to any one of the two preceding embodiments, wherein the item of identification information of the user is retrieved from the available user ID profile, specifically from the selected user ID profile.
Embodiment 25: The analytical method according to the preceding embodiment, wherein the evaluating in step i. comprises prompting the user to confirm validity of the retrieved item of identification information.
Embodiment 26: The analytical method according to the preceding embodiment, wherein, in case the user confirms validity of the retrieved item of identification information, the evaluation in step i. is affirmative.
Embodiment 27: The analytical method according to any one of the two preceding embodiments, wherein, in case the user denies validity of the retrieved item of identification information, the evaluation in step i. is negative.
Embodiment 28: The analytical method according to any one of the preceding embodiments referring to an analytical method, further comprising assigning an outcome of the analytical step to the valid item of identification information.
Embodiment 29: The analytical method according to any one of the preceding embodiments referring to an analytical method, further comprising providing at least one certificate on an outcome of the analytical step.
Embodiment 30: The analytical method according to any one of the preceding embodiments referring to an analytical method, wherein the at least one action initiated in step ii.2 further comprises performing the analytical step for personal information only, specifically without providing a certificate on an outcome of the analytical step.
Embodiment 31: A computer program comprising instructions which, when the program is executed by a mobile device having at least one camera and at least one RFID reader device cause the mobile device to perform at least one of the identification method and the analytical method according to any one of the preceding embodiments referring to an identification method or to an analytical method, respectively.
Embodiment 32: A computer-readable storage medium, specifically a non-transient computer-readable storage medium, comprising instructions which, when the instructions are executed by a mobile device having at least one camera and at least one RFID reader device cause the mobile device to perform at least one of the identification method and the analytical method according to any one of the preceding embodiments referring to an identification method or to an analytical method, respectively.

### BRIEF DESCRIPTION OF THE DRAWINGS

Further optional features and embodiments will be disclosed in more detail in the subsequent description of embodiments. Optional features may be realized in an isolated fashion as well as in any arbitrary feasible combination, as the skilled person will realize. The scope of this disclosure is not restricted by the disclosed embodiments. The embodiments are schematically depicted in the Figures. Therein, identical reference numbers in these Figures refer to identical or functionally comparable elements.

The above-mentioned aspects of exemplary embodiments will become more apparent and will be better understood by reference to the following description of the embodiments taken in conjunction with the accompanying drawings, wherein:

FIG. 1 shows an embodiment of a mobile device having at least one camera and at least one RFID reader device in a schematic view; and

FIG. 2 shows a flow chart of an embodiment of an identification method and an analytical method for detecting at least one analyte in a sample of a body fluid of a user.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

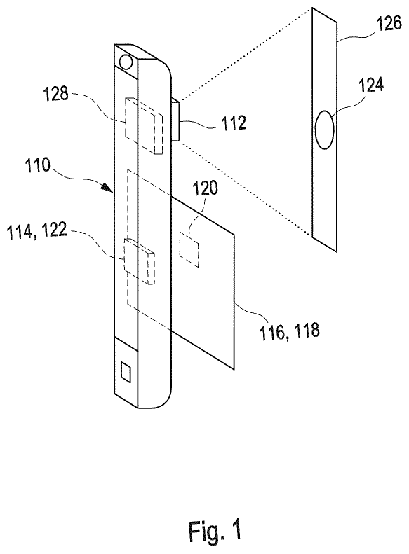

FIG. 1 shows an exemplary embodiment of a mobile device 110 having at least one camera 112 and at least one RFID reader device 114 in a schematic view. The RFID reader device 114 may be configured for reading out data from at least one RFID identifier 116. The RFID identifier 116 may, as an example, be or may comprise at least one electronic identification card 118. The RFID identifier 116 may comprise at least one RFID chip 120, specifically a 13.56 MHz RFID chip, more specifically a 13.56 MHz RFID chip using the ISO 7816 protocols. The RFID reader device 114 may be configured to read out the RFID chip 120 by using radio-frequency electromagnetic radiation, specifically when the RFID identifier 116 is in a communication range of the RFID reader device 114. The RFID reader device 114 may, as an example, comprise a near-field communication (NFC) reader device 122.

The mobile device 110 may further be configured for capturing at least one image of at least one detection portion 124 of at least one test element 126 by using the camera 112 of the mobile device 110. In the exemplary embodiment of FIG. 1, the test element 126 may be configured for performing at least one color-change detection reaction for detecting blood glucose in the sample of the body fluid, such as a blood glucose test, when a sample of a body fluid is applied to the test element 126. However, alternatively or additionally, as another example, the test element 126 may comprise at least one lateral flow assay, specifically at least one lateral flow assay configured for performing at least one reaction during which one or more optically detectable features on or within the test element 126 become visible for detecting a presence of a virus in the sample of the body fluid, such as a rapid antigen test. Various further options may also be feasible.

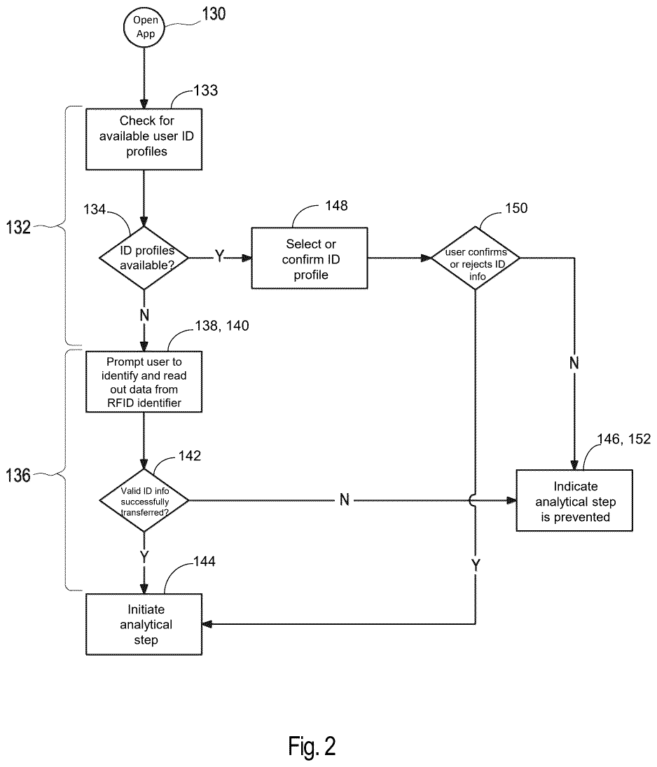

The mobile device 110 may further comprise at least one processor 128. The processor 128 may be configured, such as by software programming, to cause the mobile device 110 to perform at least one of the identification method and the analytical method according to this disclosure, such as according to the exemplary embodiment shown in FIG. 2 and/or according to other embodiment disclosed herein, respectively. For a detailed description of the identification method and the analytical method, reference is made to the description of FIG. 2.

FIG. 2 shows a flow chart of an exemplary embodiment of an identification method and an analytical method for detecting at least one analyte in a sample of a body fluid of a user. In the exemplary embodiment of FIG. 2, the identification method and the analytical method may be performed in a combined fashion. However, other embodiments in which the identification method and the analytical method are performed distinct from each other, e.g., performing the identification method with step a. to c. prior to the analytical method with steps i. and ii., are also feasible.

The identification method and the analytical method comprise using a mobile device 110, specifically a mobile communication device, the mobile device 110 having at least one camera 112 and at least one RFID reader device 114. For example, both the identification method and the analytical method may be performed using the same mobile device 110, such as the mobile device 110 in the exemplary embodiment of FIG. 1. Other options are also feasible.

The identification method and the analytical method may be initiated by the user, e.g., by the user opening an application running on the mobile device 110 (denoted by reference number 130).

The analytical method comprises step i. (denoted by reference number 132): evaluating if a validating step c. of a previously performed identification method according to this disclosure, such as according to any one of the embodiments disclosed above and/or according to any one of the embodiments disclosed in further detail below, was successful, indicating that at least one valid item of identification information of the user has been successfully transferred with the data from the RFID identifier 116 to the RFID reader device 114. Specifically, the evaluating in step i. may comprise checking for available user ID profiles, specifically user ID profiles generated by the previously performed identification method (denoted by reference number 133). In case no user ID profiles are available, the outcome of the evaluation in step i. is negative and the analytical method may proceed at decision node 134 with the path denoted by “N.”

In this example of path “N,” the outcome of the evaluation in step i. is negative and the analytical method comprises step ii.2 (denoted by reference number 136): If the evaluation in step i. is negative, specifically indicating that no valid item of identification information of the user has been successfully transferred from the user's RFID identifier 116 to the RFID reader device 114 of the user's mobile device 110, initiating performing the identification method according to this disclosure.

The identification method comprises:
a. (denoted by reference number 138) prompting the user to identify by bringing the at least one RFID identifier 116 of the user into a communication range of the RFID reader device 114;
b. (denoted by reference number 140) reading out data from the RFID identifier 116 by using the RFID reader device 114;
c. (denoted by reference number 142) validating the data read out in step b., wherein the validating comprises checking if at least one valid item of identification information of the user was successfully transferred with the data from the RFID identifier 116 to the RFID reader device 114.

In case at least one valid item of identification information of the user has been successfully transferred with the data from the RFID identifier 116 to the RFID reader device 114, step c. may comprise generating a user ID profile for the user, specifically on the mobile device 110. The user ID profile may specifically comprise the at least one valid item of identification information. Additionally or alternatively, the user ID profile may comprise at least one item of ID profile generation information on the user, the at least one item of ID profile generation information comprising information on the result of step c.. For example, the item of ID profile generation information may comprise at least one item of information indicating that the valid item of identification information of the user was successfully transferred. Additionally or alternatively, the user ID profile may comprise user-specific operational data, e.g., settings of an application running of the mobile device 110, specifically allowing performing the analytical method adjusted to the user's habits or capabilities.

In this case the analytical method proceeds at decision node 142 with path “Y,” the evaluation in step i. is affirmative, specifically indicating that at least one valid item of identification information of the user has been successfully transferred from the user's RFID identifier 116 to the RFID reader device 114 of the user's mobile device 110, and the analytical method comprises step ii.1: initiating at least one analytical step (denoted by reference number 144), the at least one analytical step 144 comprising detecting the analyte by evaluating at least one image of the at least one detection portion 124 of the at least one test element 126 having the sample of the body fluid of the user applied thereto, the at least one image being acquired with the camera 112.

In case no valid item of identification information of the user has been successfully transferred with the data from the RFID identifier 116 to the RFID reader device 114, step c. may comprise generating no user ID profile for the user. Alternatively or additionally, in case no valid item of identification information of the user was successfully transferred with the data from the RFID identifier 116 to the RFID reader device 114, the identification method may further comprise prompting the user at least one item of failure information. The item of failure information may indicate at least one of a read out of an invalid item of identification information and a failed transfer of the item of identification information. The user may be prompted to repeat performing steps a. to c.. In this case, the identification method may proceed with path “N” and may prevent the analytical step 144 (denoted by reference number 146).

Turning back to decision node 134, in case one or more user ID profiles are available, the outcome of the evaluation in step i. is positive and the analytical method may proceed at decision node 134 with the path denoted by “Y.” In case two or more user ID profiles are available, specifically two or more user ID profiles for different users, the analytical method may comprise prompting the user to select a user ID profile or to confirm a pre-selected user ID profile, specifically a pre-selected user ID profile corresponding to the user ID profile from a last login (denoted by reference number 148). The item of identification information of the user may be retrieved from the available user ID profile, specifically from the selected user ID profile. Additionally, the evaluating in step i. may comprise prompting the user to confirm validity of the retrieved item of identification information. In case the user confirms validity of the retrieved item of identification information, the evaluation in step i. may be affirmative and the analytical method, in this example, may proceed at decision node 150 with path “Y.” In case the user denies validity of the retrieved item of identification information, the evaluation in step i. may be negative and the analytical method, in this example, may proceed at decision node 150 with path “N.”

In case the analytical method proceeds at decision node 150 with path “N,” the evaluation in step i. is negative, specifically indicating that no valid item of identification information of the user has been successfully transferred from the user's RFID identifier 116 to the RFID reader device 114 of the user's mobile device 110, and the analytical method comprises step ii.2: generating at least one item of preventing information, the item of preventing information indicating that the analytical step is to be prevented (denoted by reference number 152).

In case the analytical method proceeds at decision node 150 with path “Y,” the evaluation in step i. is affirmative, specifically indicating that at least one valid item of identification information of the user has been successfully transferred from the user's RFID identifier 116 to the RFID reader device 114 of the user's mobile device 110, and the analytical method comprises step ii.1: initiating at least one analytical step (denoted by reference number 144).

The at least one analytical step 144 may further comprise, specifically prior to the evaluating of the at least one image, performing at least one of the following actions (not shown in FIG. 2):
prompting the user to apply the sample of the body fluid to the at least one test element 126;
prompting the user to confirm application of the sample of the body fluid to the at least one test element 126;
displaying at least one waiting time between sample application and capturing of the at least one image, e.g., for one or more seconds, specifically for one or more minutes, which may specifically be dependent on the test element 126, wherein during the waiting time, no image capturing may be allowed;
capturing the at least one image of the at least one detection portion 124 of the test element 126 having the sample of the body fluid of the user applied thereto using the camera 112 of the mobile device 110;
retrieving the at least one image of the at least one detection portion 124 of the test element 126 having the sample of the body fluid of the user applied thereto.

The analytical step 114 may further comprise processing the image data, e.g., one or more of a section of the image and/or at least one color value of the image, and deriving a concentration and/or a presence of the analyte in the sample of the body fluid therefrom.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

### LIST OF REFERENCE NUMBERS

110 mobile device
112 camera
114 RFID reader device
116 RFID identifier
118 electronic ID card
120 RFID chip
122 NFC reader device
124 detection portion
126 test element
128 processor
130 start
132 evaluating if a validating step c. of a previously performed identification method was successful
133 checking for available user ID profiles
134 decision node
136 performing the identification method
138 prompting the user to identify
140 reading out data from the RFID identifier
142 validating the data
144 analytical step
146 prevent the analytical step
148 prompting the user to select or confirm a user ID profile
150 decision node
152 generating at least one item of preventing information

What is claimed is:

1. A method for detecting an analyte in a sample of a body fluid, the analytical method comprising using a mobile device having a camera and an RFID reader, the analytical method comprising:
i. evaluating if a validating step c. of a previously performed identification method was successful, indicating that a valid item of identification information of a user has been successfully transferred with the data from an RFID identifier to the RFID reader, the identification method being a computer-implemented identification method of identifying a user for an analytical method of detecting at least one analyte in a sample of a body fluid, the identification method comprising:
a. using the mobile device to prompt the user to identify by bringing an RFID identifier of the user into a communication range of the RFID reader;
b. reading out data from the RFID identifier by using the RFID reader;
c. validating the data read out in step b., wherein the validating comprises checking if a valid item of identification information of the user was successfully transferred with the data from the RFID identifier to the RFID reader, wherein, in step c., the checking of the data for a valid item of identification information on the user transferred with the data from the RFID identifier to the RFID reader comprises checking if the user is authorized to perform the analytical method;
ii. depending on the outcome of step i., taking one of the following actions:
ii.1. if the evaluation in step i. is affirmative, initiating at least one analytical step, the at least one analytical step comprising detecting the analyte by evaluating at least one image of at least one detection portion of at least one test element having the sample of the body fluid of the user applied thereto, the at least one image being acquired with the camera;
ii.2. if the evaluation in step i. is negative, initiating at least one action selected from the group consisting of:
performing the identification method;
generating at least one item of preventing information, the item of preventing information indicating that the analytical step is to be prevented;
wherein step i. comprises checking for available user ID profiles, wherein, when two or more user ID profiles are available, the analytical method comprises prompting the user to select a user ID profile or to confirm a pre-selected user ID profile, wherein the item of identification information of the user is retrieved from the available user ID profile, wherein step i. comprises prompting the user to confirm validity of the retrieved item of identification information, wherein, in case the user confirms validity of the retrieved item of identification information, the evaluation in step i. is affirmative; and
wherein the readout in step b. comprises a transmission of the at least one item of identification information from the RFID identifier to the RFID reader in an encoded format, wherein the encoded format comprises the use of a Hamming code.

2. The analytical method according to claim 1, further comprising providing a certificate on an outcome of the analytical step.

3. The analytical method according to claim 1, wherein the action initiated in step ii.2 further comprises performing the analytical step for personal information only.

4. The analytical method according to claim 1, wherein, when at least one valid item of identification information of the user has been successfully transferred with the data from the RFID identifier to the RFID reader, step c. comprises generating a user ID profile for the user, wherein the user ID profile comprises the at least one valid item of identification information.

5. The analytical method according to claim 4, wherein the user ID profile comprises at least one item of ID profile generation information on the user, the at least one item of ID profile generation information comprising information on the result of step c.

6. The analytical method according to claim 1, wherein, when no valid item of identification information of the user was successfully transferred with the data from the RFID identifier to the RFID reader, the identification method further comprises prompting the user an item of failure information that indicates at least one of a read out of an invalid item of identification information and a failed transfer of the item of identification information.

7. The analytical method according to claim 1, wherein, in step c., the checking of the data for at least one valid item of identification information on the user transferred with the data from the RFID identifier to the RFID reader further comprises at least one of:
checking if the at least one item of identification information on the user was successfully and completely transferred with the data from the RFID identifier to the RFID reader;
checking if the at least one item of identification information on the user transferred with the data from the RFID identifier to the RFID reader is sufficient;
checking if the at least one item of identification information on the user matches at least one previously registered item of identification information on the user;
checking if the at least one item of identification information on the user was transferred from the RFID identifier to the RFID reader within at least one admissible region.

8. The analytical method according to claim 1, wherein the readout in step b. comprises a secure data transmission of the item of identification information from the RFID identifier to the RFID reader.

9. The analytical method according to claim 1, wherein step c. comprises determining at least one checksum of the data transferred from the RFID identifier to the RFID reader.

10. A computer program comprising instructions which, when the program is executed by a mobile device having at least one camera and at least one RFID reader, cause the mobile device to perform the analytical method according to claim 1.

11. A computer-readable storage medium comprising instructions which, when the instructions are executed by a mobile device having at least one camera and at least one RFID reader, cause the mobile device to perform the analytical method according to claim 1.
